(19) United States
(12) Patent Application Publication
(10) Pub. No.: US 2022/0098330 A1
(43) Pub. Date: Mar. 31, 2022

(54) METHOD FOR THE MANUFACTURING OF CHITIN DERIVATIVES THROUGH TREATMENT WITH ULTRASOUNDS

(71) Applicant: TEXOL S.R.L., Alanno (IT)

(72) Inventor: Fabio DI BERARDINO, Roccamontepiano (IT)

(73) Assignee: TEXOL S.R.L., Alanno (IT)

(21) Appl. No.: 17/310,098

(22) PCT Filed: Jan. 15, 2020
(86) PCT No.: PCT/IB2020/050304

§ 371 Date: Jul. 16, 2021

(30) Foreign Application Priority Data
Jan. 17, 2019 (IT) 102019000000749

(51) Int. Cl. C08B 37/08; A61K 8/73; A61Q 19/00; A61K 8/02

(52) U.S. Cl. CPC C08B 37/003; A61K 8/736; A61K 2800/413; A61K 8/027; A61Q 19/00

(57) ABSTRACT

The invention relates to a new process for the manufacturing of chitin derivatives, in particular of nanofibrillary chitin, sustainable from the industrial implementation point of view and suitable to produce chitin derivatives having improved properties. The invention further relates to chitin derivatives obtainable with such method. The invention further relates to the uses of such derivatives which comprise for example the formulation of pastes and aqueous gels useful for the topic applications on skin, the manufacturing of materials of biomedical interest, as well as the deposition of chitin nanofibrils on coating surfaces or the incorporation of the same inside articles such as, for example, hygiene sanitary products.

### TECHNICAL FIELD OF THE INVENTION

The invention relates to a new process for the manufacturing of chitin derivatives, in particular nanofibrillary chitin, sustainable from the industrial implementation point of view and suitable to manufacture chitin derivatives having improved properties. The invention further relates to chitin derivatives obtainable with such method. The invention further relates to the uses of such derivatives which comprise for example the formulation of pastes and aqueous gels useful for the topic applications on skin, the manufacturing of materials of biomedical interest, as well as the deposition of chitin nanofibrils on coating surfaces or the incorporation of the same inside items such as, for example, hygiene sanitary products.

### STATE OF PRIOR ART

Previous experiments showed the utility of chitin nanofibrils and other chitin derivatives in several applications, for example as reinforcing material in textile fibres, in cosmetics e in therapeutic treatments. Native chitin for example can be found in crustaceans as microfibril material in alpha polymorphic form, having a little degree of deacetylation given by the enzyme chitin deacetylase with the purpose of binding it to the proteins during biosynthesis. In vitro, the partial acid hydrolysis takes place more quickly on the less crystalline portions of chitin fibres, thus releasing nanofibrils; these are stiff, resistant objects, having an average length of 250 nm with rectangular section of about 7×20 nm. The surface of one gram of nanofibrils is of 180 m².

The known methods for manufacturing chitin nanofibrils or other derivatives thereof have disadvantages, for example the processes having thermal treatments lead to the even irreversible aggregation of nanofibrils and a low yield.

Other methods instead can be used with small amounts on laboratory scale, they are not suitable to be extended to manufacturing on a larger scale.

The object of the present invention is to provide a solution to the problems left unsolved by the known processes, above all that of the difficult applicability at the industrial level of the known methods and that of the difficult redispersion in aqueous means of the chitin derivatives manufactured with the known methods with a high yield.

### SUMMARY OF THE INVENTION

The inventors of the present patent application have developed a new process for the manufacturing of chitin derivatives, in particular of nanofibrils. The process is based upon the use of ultrasound apparatuses allowing to obtain the chitin derivatives with reduction of time, increase in yields and generally by using milder reaction conditions. In particular, lower concentrations of aggressive reagents, such as acids and in some cases, only water, can be used.

Another advantage of the ultrasound process, in some cases/applications is obtained by treating directly the biomass containing chitin so as to disaggregate it. In this way the use of acids can be avoided, by avoiding that the carbon dioxide, coming from the calcium carbonate existing in huge amounts in some biomasses, such as crustaceans' residues and insects' cuticles, enters the atmosphere. In this way, one succeeds in using the biomass both as such (for example as agricultural fertilizer) and in proceeding with additional purifications of the chitin derivatives (for example to be used as bio-pesticide, cosmetic use, medical use), with a process having a minimum environmental impact. Then, in the light of what said, in the first place the invention relates to a process for the manufacturing of chitin derivatives, in particular chitin nanofibrils, comprising the following steps:

i) dispersing in an acid solution chitin, chitosan and/or biomass comprising chitin and/or chitosan;
ii) subjecting the dispersion prepared in step i) to one or more sonication steps.

In the second place, the invention relates to chitin derivatives, in particular chitin nanofibrils, obtainable through the process of the invention.

In the third place, the invention relates to chitin derivatives obtainable through the process of the invention to be used in a therapeutic, surgical or cosmetic treatment, in particular in protective or healing treatments of abrasions, wounds, skin burns, or in treatments for supporting and stimulating the processes of haemostasis, healing and regeneration of the damaged tissues, or in dermocosmesis treatments through hypodermic subcutaneous injection or through cutaneous application with masks, films or sponges.

In fifth place, the invention relates to compositions comprising chitin derivatives obtainable in a form selected from the solid state, aqueous liquid or organic suspension, gel, paste, or other form suitable for application on skin or mucous membranes and a pharmacologically or cosmetically acceptable excipient, optionally comprising additional components selected from thickeners, plasticizers, emulsifiers, preservatives, bactericides, fungicides, antimicrobials, immunomodulatory agents, metal ions, alpha amino acids, beta amino acids, carotenoids or antioxidants of any origin, solar filters, vegetable extracts, moisturizers, derivatives or mixtures of the same and other active principles having known pharmacological activity.

The invention further relates to items selected from films, sponges, synthetic or natural fabric, hygiene sanitary products comprising chitin derivatives, in particular chitin nanofibrils, obtainable through the process of the invention.

At last, the invention also relates to any one of the above-mentioned uses of chitin derivatives obtainable through the process of the invention.

### DESCRIPTION OF THE FIGURES

FIG. 1: the figure illustrates the preparation of the dispersion step in a thermostated balloon equipped with reflux tube.

FIG. 2: FIG. 2A illustrates a dispersion obtained with the process of prior art (example 1), whereas FIG. 2B a dispersion obtained with an embodiment of the present invention (example 2).

FIG. 3: FIG. 3A illustrates a dispersion obtained with the process of prior art (example 1), whereas FIG. 3B a dispersion obtained with an embodiment of the present invention (example 3).

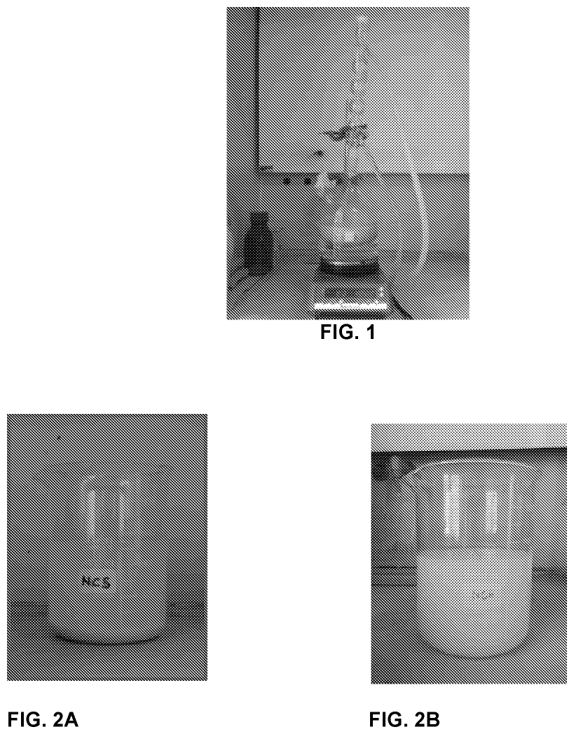

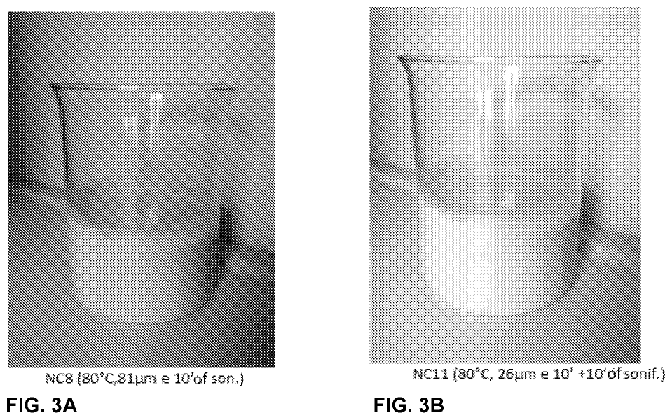

### DETAILED DESCRIPTION OF THE INVENTION

In an aspect the present invention relates to a process for the manufacturing of chitin derivatives, in particular for the manufacturing of chitin nanofibrils and colloidal chitosan.

The process provides a step i) wherein in an acid solution chitin, chitosan and/or biomass comprising chitin and/or chitosan are dispersed.

The starting product which is dispersed then could be pure chitin or chitosan, for example grab alpha-chitin, available on the market in form of powder or flakes. Other types of chitin can equally be used, for example the biomass comprising chitin obtained from crustaceans' residues and insects' cuticles.

Or chitosan which is a derivative of chitin, a polysaccharide existing in the exoskeleton of invertebrates such as insects, grabs and shrimps. Through a hydrolysis reaction of the amide bonds of chitin (with transformation of the amide bonds into amino groups) chitosan is obtained from chitin. Chitosan mainly is characterized by parameters such as: molecular weight, deacetylation degree, possible salification of the amino groups.

According to the process of the present invention by acting starting from pure chitin or chitosan colloidal dispersions are obtained (in case of chitin, chitin nanofibrils are obtained). Such aqueous dispersions can be used in cosmetic, medical field or as bio-pesticides in agriculture.

According to the process of the present invention by acting with pure chitin or chitosan and, apart from ultrasounds, by using more drastic reaction conditions such as use of strong acids or alkali or by using enzymes one succeeds in obtaining other chitin and chitosan derivatives such as the oligosaccharide derivatives which are considered to have an increased antimicrobial power. In this case the ultrasound process allows better yields, shorter time, milder reaction conditions.

According to the process of the present invention by acting with biomasses such as crustacean shells or fungal biomasses aqueous dispersions are obtained including, apart from chitin, polypeptides, mineral salts and calcium carbonate. Such materials can be used in agriculture as fertilizers and bio-pesticides.

In step i) of the process the powder of chitin, chitosan and/or biomass comprising them are dispersed in an acid, preferably aqueous, solution.

Preferably it is added to water before the material comprising chitin, for example pure chitin in power and then the acid.

The amount of chitin, chitosan and/or biomass comprising them added in solution is preferably in a concentration by weight with respect to the final acid solution ranging between 0.5 and 20% w/w, still more preferably between 2 and 10% w/w, still more preferably between 3 and 6% w/w.

The solution is acidified with strong acid, preferably HCl 3M 37% HCl.

The solution pH will be for example at least pH 3, w/w preferably at least about 2, that is for example 2.1, 2.2 etc.

The amount of strong acid, for example 37% HCl, in solution is preferably in a concentration by weight with respect to the final acid solution between 5 and 35% w/w, still more preferably between 10 and 30%, still more preferably between 10 and 15%.

The dispersion will be kept under stirring at least at 50° C., preferably between 70° C. and 100° C., for at least minutes, preferably up to 5 hours, still more preferably between 30 minutes and 3 hours.

Before the sonification step the solution will be preferably cooled down for example until room temperature (15-35° C.)

The solution will be prepared for example in a thermostated balloon equipped with reflux tube.

In the subsequent step ii) the solution is subjected to one or more sonication steps.

According to an embodiment said one or more sonication steps are performed at a power of at least 260 W. For productions at industrial level with higher volumes one could proceed with ultrasound transducers having higher power or acting with a “cascade” of transducers, as it is known from the state of art.

Said one or more sonication steps are performed for example for a total time ranging between 2 and 30 minutes, in particular each sonication step is performed for a maximum time of 2 minutes by leaving cooling at room temperature (15-30° C. or however not higher than 50° C.) the dispersion before the next sonication step.

The used sonotrode width for example will be ranging between 10 and 100 μm. The process could provide subsequent steps wherein the sonicated dispersion is subsequently subjected to additional passages, for example spray-drying passages known in the state of art.

The chitin derivatives obtainable by means of the herein described process, in particular the chitin nanofibrils, can be used in several cosmetic and therapeutic applications.

In particular they can be used in the protective or healing treatment of abrasions, wounds, skin burns, in particular in the treatment for supporting and stimulating the processes of haemostasis, healing and regeneration of the damaged tissues.

The chitin derivatives of the invention can further be used in the field of cosmetic surgery, in particular dermocosmesis, as skin fillers for the treatment of wrinkles and other cutaneous irregularities and imperfections through hypodermic subcutaneous injection or in the cutaneous treatment through masks, films, sponges.

An additional application of chitin derivatives of the invention relates to the manufacturing of films and fibres and other solid objects. The incorporation of nanofibrils is performed through known techniques, that is spinning (melt-spinning, dry-spinning or wet-spinning), extrusion (coextrusion, filming from solutions, calendering) and hot forming.

Other application fields are for example:
Upper layer (in contact with derma) of hygiene sanitary products such as: napkins for children, female sanitary towels, products for incontinence
in medical products as product portions or as product on itself (for example bandages). Such products can be used for the treatment of wounds and burns
Beauty masks for cosmetic use
Packaging of food products
Fertilizers in agricultural field
Bio-pesticides in agricultural field

### EXAMPLES

The synthesis of chitin nanofibrils with ultrasound process according to a preferred embodiment of the present invention and the comparison with the processes of known art are reported hereinafter. The chitin used in all experiments was obtained from the firm: Jiangsu Aoxin Biotechnology Co. Ltd. The chitin batch number is 20160801. As physical form the material appeared in a powder, >90% thereof passing through a 100-mesh filter.

### Example 1: Chitin Nanofibrils Manufactured According to the Process of Prior Art

20 g of chitin are dispersed in 265 g of distilled water. 135 g of 37% HCl (chitin concentration 5% w/w) are added, the whole in a thermostated balloon equipped with reflux tube.

The sample is heated under stirring at 70° C. for 3 hours and then it is left to cool down for 3 hours and the sample is transferred into beakers for additional tests and measurements. Such sample was marked as NCS.

### Example 2: Chitin Nanofibrils Manufactured According to the Process with Ultrasounds According to an Embodiment of the Present Invention and the Same Amount of Acid of Example 1

The same chitin sample used in the process described in example 1 was used. The treatment with ultrasound was performed by using a sonificator Hielscher UIP 1000 HD used at the power of 260 W. The treatment with ultrasounds was performed for 2 minutes and the sample was then left to cool down for 3-5 minutes so as not to make the solution to boil. The samples NC1, NC2, NC3, NC6 were thus manufactured, characterized by a total time of treatment with ultrasounds respectively of: 10′, 2′, 20′, 30′.

TABLE 2
Summary of test results

| / | NC1 | | NC2 | | NC3 | | NC5 | | NC6 | |
|---|---|---|---|---|---|---|---|---|---|---|
| ACID | 3 | h | 3 | h | 3 | h | 3 | h | 3 | h |
| HYDROLYSIS | | | | | | | | | | |
| TIME | | | | | | | | | | |
| TEMPERATURE | 80° | C. | 80° | | 80° | | 80° | | 80° | |
| AMOUNT | 135 | g | 135 | g | 135 | g | 135 | g | 135 | g |
| OF HCl | | | | | | | | | | |
| SONOTRODE | 26 | μm | 26 | μm | 26 | μm | 26 | μm | 26 | μm |
| WIDTH | | | | | | | | | | |
| SONIFICATION | 10′ | | 2′ | | 20′ | | no | | 30′ | |
| TIME | | | | | | | | | | |
| pH SUSPENSION | 1 < pH < 2 | | 1 < pH < 2 | | 1 < pH < 2 | | 1 < pH < 2 | | 1 < pH < 2 | |
| DRY RESIDUE | 7.95 | g | 13.2 | g | 5.29 | g | 15.25 | g | 4.95 | g |
| WEIGHT | | | | | | | | | | |
| H2O VOLUME | 3.6 | l | 2.8 | l | 10.8 | l | 5.8 | l | 11 | l |
| WASHES | | | | | | | | | | |

| | | | | | |
|---|---|---|---|---|---|
| % YIELD | ≤60% | ≤34% | ≤73% | ≤23% | ≤75% |

The dry residue (acid solution left to decant one night) in the samples subjected to sonication decreases and this is already an indication of increased yield of conversion into chitin nanofibrils. Even by observing the samples, in particular those subjected to most intense sonication cycles it is possible to observe a different aspect (FIGS. 2A and 2B).

### Example 3: Process with Ultrasounds Acid Amount Reduced by 50% with Respect to Example 1

In this case 265 g of distilled water and 61.5 g of 37% HCl (the concentration of chitin was 6% w/w) were added to 20 g of chitin. At the end of decantation, the samples were washed with water (4 washings) until obtaining a pH of about 2.0. Such samples were then analysed and characterized as shown in example 4. The table below summarizes the main features of the samples:

TABLE 1
Summary of tests

| / | NC1 | | NC2 | | NC3 | | NC5 | | NC6 | |
|---|---|---|---|---|---|---|---|---|---|---|
| ACID | 3 | h | 3 | h | 3 | h | 3 | h | 3 | h |
| HYDROLYSIS | | | | | | | | | | |
| TIME | | | | | | | | | | |
| TEMPERATURE | 80° | C. | 80° | | 80° | | 80° | | 80° | |
| AMOUNT OF HCl | 50% | | 50% | | 50% | | 50% | | 50% | |
| SONOTRODE | 26 | μm | 26 | μm | 81 | μm | 81 | μm | 26 | μm |
| WIDTH | | | | | | | | | | |
| SONIFICATION | 10′ | | 20′ | | 10′ | | 20′ | | 10′ + 10′* | |
| TIME | | | | | | | | | | |
| pH SUSPENSION | 1 < pH < 2 | | 1 < pH < 2 | | 1 < pH < 2 | | 1 < pH < 2 | | 1 < pH < 2 | |
| DRY RESIDUE | 10.52 | g | 8.42 | g | 12.95 | g | 10.13 | g | 8.06 | g |
| WEIGHT | | | | | | | | | | |
| H2O VOLUME | 7.5 | l | 10.5 | l | 12 | l | 13.2 | l | 10.5 | l |
| WASHINGS | | | | | | | | | | |

| | | | | | |
|---|---|---|---|---|---|
| % YIELD | 47.4% | 57.9% | 35.2% | 49.3% | 59.97% |

*10′ of pre-sonification of the solution, before the acid hydrolysis reaction and 10′ of sonification after the reaction.

Even in this case some samples show a low level of material precipitation, which means that the reaction performance has improved with respect to the reference (NC 5):

### Example 4: Characterization of the Samples Manufactured in Examples 1-3

The samples were then subjected to additional purification through cycles of washing in water until obtaining a pH of about 2.0. On such samples, analyses were performed through FT-IR spectra executions, Particle size analysis, XPS (X-ray photoelectronic spectroscopy), SEM electronic microscopy.

In particular XPS measures show that the surface of the obtained nanofibrils is widely deacetylated (that is the chitin amide groups were transformed into amino groups). The samples marked as “MAVI” and “TEXOL” were obtained according to the process of prior art (simple acid hydrolysis).

TABLE 2
acetylation degree and percentage of charged nitrogen, estimated
from the curve fitting of the analysed samples.

| Sample | Acetylation degree (%) |
|---|---|
| NC MAVI | 12 |
| NC TEXOL | 16 |
| NC1 mix | 9 |
| NC2 mix | 9 |
| NC3 mix | 7.9 |

As far as the particle size analysis is concerned, the analyses shown on the tables below underlined that sonication leads to a considerable decrease in particles' sizes and that already 10 minutes are sufficient to obtain significative results. X99 value further allows to state that the maximum size of the analysed particles of the several samples is 90 micrometres for MAVI, 98 micrometres for TEXOL (both of them prior art) and 29.7 micrometres for NC1 sample.

TABLE 3
PSA results

| [symbol] | MAVI | | | TEXOL | | | NC1 | | | NC2 | | | NC3 | | |
|---|---|---|---|---|---|---|---|---|---|---|---|---|---|---|---|
| [symbol] [μm] | [symbol] .23 | 6.04 | 5. [symbol] 3 | 3.93 | 3.89 | 3.85 | 2.1 | 2.05 | 2.03 | 3.83 | 3.75 | 3.71 | 2. [symbol] 4 | 2.1 | 1.95 |
| [symbol] [μm] | 24.32 | 23.59 | 22.63 | 15.24 | 15.14 | 14.88 | 5.8 | 5.53 | 5.41 | 1 [symbol] .99 | 15.8 | 15. [symbol] | 7.3 | 6.52 | 6.01 |
| [symbol] [μm] | [symbol] 1.96 | 60.3 [symbol] | 56.5 [symbol] | 52.4 | 53.05 | 50.66 | 16.3 | 15.01 | 14.71 | 3 [symbol] .2 [symbol] | 35. [symbol] 1 | 34.84 | 17.1 [symbol] | 15.19 | 14.0 [symbol] |
| [symbol] [μm] | 93.6 [symbol] | [symbol] 1.6 | [symbol] 4.9 [symbol] | 100.55 | [symbol] 7. [symbol] 7 | 95. [symbol] | 30.13 | [symbol] 8.95 | 29.9 | 62.57 | 57.9 | 56.93 | 29.7 [symbol] | 27.34 | 25.35 |
| VMD [μm] | 29.61 | 2 [symbol] .7 [symbol] | 27.25 | 22.6 [symbol] | 22.55 | 21.93 | 7.74 | 7.29 | 7.17 | 18.57 | 18.13 | 17.77 | 8.78 | 7.82 | 7.23 |

[symbol]

indicates data missing or illegible when filed

TABLE 4
mean values of PSA results

| / | MAVI | TEXOL | NC1 (10 [symbol] ) | NC2 (2 [symbol] ) | NC3 (2 [symbol] ) |
|---|---|---|---|---|---|
| X10 (μm) | 5.03 | 3.89 | 2.06 | 3.76 | 2.13 |
| X50 (μm) | 23.51 | 15.09 | 5.58 | 15.7 [symbol] | 6.6 [symbol] |
| X [symbol] (μm) | 59.62 | 52.04 | 15.34 | 35.5 | 15.49 |
| X39 (μm) | 90.09 | 9 [symbol] .03 | 29. [symbol] | 5 [symbol] .13 | 27.48 |
| VMD (μm) | 28.5 [symbol] | 22.3 [symbol] | 7.4 | 18.1 [symbol] | 7. [symbol] 4 |

[symbol]

indicates data missing or illegible when filed

### Example 5: Colloidal Chitosan Manufactured with Ultrasounds

U.S. Pat. No. 9,173,393 B2 describes a method for manufacturing an insoluble chitosan powder able to limit the growth of fungus Brettanomyces in fermented liquids (such as for example wine). In particular the obtained chitosan in basic form is subjected to the action of micronizer mills (procedure described on page 11 of the patent) until obtaining a powder of chitosan with particles of 5-50 microns. It is believed that such micropowder is much more active than the normal chitosan powder due to the reduced sizes of the particles which would increase the active surface thereof.

According to the process of the present invention a dispersion of 10% w/w of chitosan in basic form (provided by the firm Dounghness, Seattle, USA) in water is subjected to sonication by using the device Hielscher UIP 1000 HD with a total sonication time of minutes (mode analogous to what described in example 2). At the end the sample appears as a colloidal solution. The solution is dried (according to what illustrated in the U.S. Pat. No. 8,552,164 B2) through spray drying with Buchi mini-90 apparatus.

1. A process for the manufacturing of chitin derivatives comprising the following steps:
i) dispersing chitin, chitosan and/or biomass comprising chitin and/or chitosan in an acid solution; and
ii) subjecting the dispersion prepared in step i) to one or more sonication steps.

2. The process according to claim 1 comprising the following steps:
i) dispersing in an acid solution chitin, chitosan and/or biomass comprising chitin and/or chitosan;
ii) maintaining said dispersion under agitation at least at 50° C. for at least 5 minutes,
iii) cooling said dispersion, and
iv) subjecting said dispersion prepared in step iii) to one or more sonication steps.

3. The process according to claim 1, wherein said one or more sonication steps are performed at a power of at least 260 W.

4. The process according to claim 1, wherein said one or more sonication steps are performed for a total time between 2 and 30 minutes.

5. The process according to claim 1, wherein said acid solution is prepared by adding in water a strong acid so as to obtain a pH lower than or equal to about 2.

6. The process according to claim 1, wherein said acid solution is prepared by adding in water a strong acid in a concentration by weight with respect to the final acid solution between 5 and 35% w/w.

7. The process according to claim 1, wherein said chitin, chitosan and/or biomass is added in solution in a concentration by weight with respect to the final acid solution between 0.5 and 20% w/w.

8. The process according to claim 1, comprising an additional step wherein said dispersion is subjected to a spray-drying step.

9. Chitin derivatives obtainable according to the process of claim 1.

10. The chitin derivatives according to claim 9, wherein said derivatives are nanochitin fibrils and/or colloidal chitosan.

11. A composition comprising the chitin derivatives according to claim 9 in a form selected from the dry state, aqueous or organic liquid suspension, gel, paste, or other form suitable for application on skin or mucous membranes and a pharmacologically or cosmetically acceptable excipient and comprising additional components selected between thickeners, plasticizers, bactericides, fungicides, antimicrobials, metal ions, alpha amino acids, beta amino acids, carotenoids, moisturizers, derivatives or mixtures thereof.

12. A film, sponge, or synthetic or natural fabric comprising inside thereof or adsorbed on the surface thereof the chitin derivatives according to claim 9.

13. The process according to claim 2, wherein the dispersion is maintained under agitation at between 70° C. and 100° C., for between 30 minutes and 5 hours and the cooling of the dispersion occurs until room temperature (15-35° C.) is reached.

14. The process according to claim 4, wherein each sonication step is performed for a maximum time of 2 minutes allowing the dispersion to cool down to room temperature (15-35° C.) before the next sonication step.

15. The process according to claim 5, wherein the strong acid is 37% HCl.

16. The process according to claim 6, wherein water is added in a concentration by weight with respect to the final acid solution between 10 and 30% or between 10 and 15%.

17. The process of claim 7, wherein the chitin, chitosan and/or biomass is added in a concentration by weight with respect to the final acid solution between 2 and 10% or between 3 and 6% w/w.
